(19) United States
(12) Patent Application Publication
(10) Pub. No.: US 2023/0112349 A1
(43) Pub. Date: Apr. 13, 2023

(54) Air filtration material, device for decontaminating air by filtration and manufacturing method.

(71) Applicant: CELLULOTECH INC, Victoria (CA)

(72) Inventor: Daniel SAMAIN, TOULOUSE (FR)

(21) Appl. No.: 17/914,254

(22) PCT Filed: Mar. 23, 2021
(86) PCT No.: PCT/EP2021/057432

§ 371 Date: Sep. 23, 2022

(30) Foreign Application Priority Data
Mar. 27, 2020 (FR) FR2003078

(51) Int. Cl. B01D 39/18; B32B 7/14; B32B 29/00; B32B 29/06; A62B 23/02; A61L 9/014; D21H 11/20; D21H 27/08; B32B 37/18; B32B 37/12

(52) U.S. Cl. CPC B01D 39/18; B32B 7/14; B32B 29/005; B32B 29/06; A62B 23/025; A61L 9/014; D21H 11/20; D21H 27/08; B32B 37/18; B32B 37/1292; B01D 2239/0428; B01D 2239/0421; B01D 2239/1291; B01D 2239/083; B01D 2239/10; B01D 2239/0668; B32B 2250/26; B32B 2262/062; B32B 2307/728; B32B 2307/73; B32B 2307/724; B32B 2571/00; A61L 2209/22; A61L 2209/14; B01D 2239/0208; B32B 2317/12; B32B 2260/04; B32B 2260/028

(57) ABSTRACT

The invention relates to a filtering material permeable to air and impermeable to oral, nasal and/or ocular human liquid aqueous excretions, comprising at least one bundle, referred to as a hydrophobic bundle, of at least two hydrophobic sheets which are superimposed and each formed by porous paper, having a gsm substance less than 30 g/m², each hydrophobic sheet being formed by cellulose fibres, referred to as cross-linked cellulose fibres, which are connected to each other by hydrogen bonds and by covalent bonds which are formed with at least one group of cross-linking atoms, characterised in that at least some of the hydroxyls of the cross-linked cellulose fibres which are not engaged in hydrogen bonds and which are accessible to the gases form a covalent bond with an acyl group with a hydrophobic chain.

The invention relates to a material, termed a filtering material, for the filtration of a flow of breathed air and to a method for the manufacture of a filtering material of this type.

The invention also relates to a device for the decontamination of a flow of contaminated air by filtration, the decontamination device comprising a filtering material of this type. Thus, the invention relates to a filtering material of this type that is permeable to air and impermeable to human oral, nasal and/or ocular aqueous liquid excretions—in particular to those aqueous excretions carrying pathogenic microorganisms, in particular infectious viral particles.

Epidemics and pandemics in general, and in particular the pandemic linked to the “SARS-CoV-2” coronavirus raging in 2020, pose a grave danger, not only in terms of public health on a planetary scale, but also in economic terms.

The rapid emergence of such epidemics and/or pandemics does not give the health industry enough time for the preparation and launch onto the market of drugs or vaccines that can effectively combat the pathogenic agents responsible for these epidemics/pandemics. Thus, it is vital for means to be developed in order to be able to effectively combat dissemination of these pathogenic agents. Clearly, controlling an epidemic/pandemic of this type involves complying with simple health provisions such as regularly washing hands, prohibiting contact between individuals, using disposable tissues and containment of them after use.

Although they concern the dissemination of highly contagious viral pathogenic agents, such combat measures also fall into the category of combat measures against the transmission of these pathogenic viruses between individuals, in particular between an individual carrying the pathogenic virus and a healthy individual. Although they concern the dissemination of pathogenic viruses of the respiratory tract, such combat measures essentially fall into the category of means for the filtration of a flow of air exhaled by an individual infected with the pathogenic virus and filtration of a flow of air inhaled by a healthy individual in the presence of an individual infected with the pathogenic virus.

The control of an epidemic caused by a virus targeting the respiratory tract which is itself highly contagious and against which there is neither a vaccine nor an effective drug combat measure necessarily involves establishing preventive health measures aimed at limiting contacts between individuals, among whom individuals who are infected and contagious are necessarily present. Such health measures include establishing a minimum safety distance to be complied with between the individuals. Such health measures also involve minimizing the risks of exchange between contaminated individuals and healthy individuals—by inhalation/exhalation of air, which is a vector for human oral, nasal and/or ocular aqueous liquid excretions in the form of microdroplets or macrodroplets that could contain an infectious pathogenic agent.

Pathogenic viruses of the respiratory tract of this type are disseminated by any infected individual in the form of an aerosol formed by the exhaled air and aqueous liquid particles in the form of microdroplets or macrodroplets in suspension in the exhaled air. This may be air exhaled by simple spontaneous respiration. This may also be air exhaled when speaking, when sneezing or during an episode of coughing. However, whatever the conditions for the emission of an aerosol of this type, the aqueous liquid particles in suspension in the exhaled air are loaded with infectious viral particles that could be transmitted to a healthy individual who is passing or who is within range of an infected individual who is necessarily releasing an aerosol of this type.

Wearing a protective mask, in particular a surgical mask, the aim of which is to prevent the transmission of germs carried by the surgeon to a patient as much as possible and which is fitted over the mouth and nose of the infected. individual and/or healthy individuals, is recommended.

A mask of this type, however, has a limited efficiency over time because the filtering material rapidly becomes saturated. Furthermore, a mask of this type is generally hydrophilic and the moisture retained by a mask of this type deteriorates the mechanical strength of the mask and its filtering properties. Furthermore, it is not possible to increase its mechanical strength by increasing its thickness without reducing its per to air and without altering the respiration of the wearer of that mask.

Furthermore, in the case of disposable surgical masks, the problem with their saturation and their detetioration—in particular by moistening the filtering material—during a surgical intervention can be solved by replacing the deteriorated mask with a fresh, functional mask outside the surgery area; following this replacement, the surgical intervention can then continue under good sanitary conditions. However, this cannot be the case with a filtering material used by an individual in the context of an epidemic, in which the replacement of a used filtering material by a fresh filtering material could involve contamination of the individual.

Thus, a mask solution which could enable efficient and long-term filtration and maintain respiratory comfort would be desirable. In addition, a mask solution wherein the wet strength was increased would also be desirable.

In the context of the development of such epidemics and/or pandemics, it would also be desirable for the filter masks to be capable of being fabricated easily by an individual if the need arose for personal protection or for the protection of their nearest and dearest. In this regard, the non-availability of masks for everybody may be the cause of contaminations that could turn out to be fatal. It is well known that in order to block the development of an epidemic/pandemic, it is necessary to interrupt the chains of contamination, and accessibility to a sufficient number of filtering masks for the entire population is essential. Since a pandemic is by definition global, it is also necessary to be able to prepare filtering masks cheaply so that the most disadvantaged of populations can have access to them.

The aim of the invention is to overcome all of these disadvantages.

The aim of the invention is to propose a filtering material and a device for the decontamination of air by filtration and self-protection of an individual against an atmosphere contaminated by a pathogenic microorganism, in particular by a virus.

The invention therefore proposes a filtering material and a device for the decontamination of air inhaled by an individual and for the protection of the individual wearing the decontamination device. In particular, the invention proposes a filtering material of this type and a device of this type for the decontamination of a flow of air before it is inhaled by an individual.

But the invention also proposes a filtering material and a device for the decontamination of air and for the protection of other parties vis-à-vis an individual infected with a pathogenic microorganism, in particular by a virus. In particular; the invention proposes a filtering material and a device for the decontamination of a flow of air formed by exhalation.

Thus, the invention also proposes a filtering material and a device for the decontamination, by filtration, of the air exhaled by an individual and for the protection of third parties who are not wearing a decontamination device.

The invention proposes a filtering material and a device for the decontamination of air by filtration, comprising a filtering material of this type which performs well and is comfortable.

The invention proposes a filtering material and a disposable device for the decontamination of air by filtration.

The invention proposes a filtering material and a device for the decontamination of air by filtration which can be destroyed after use in a simple, economic, safe and environmentally friendly manner.

The invention proposes a filtering material and a device for the decontamination of air by filtration with a low manufacturing cost.

The invention also proposes a filtering material and a device for decontamination by filtration that is inexpensive to destroy.

In particular, the invention provides a filtering material of this type and a decontamination device of this type that can be disposed of after use and that can be destroyed by incineration.

The invention also proposes a filtering material of this type, a device for decontamination by filtration of this type and a method for the production of a filtering material of this type that can be made by hand.

The invention also proposes a method for the one-off production of a filtering material of this type and of a decontamination device of this type.

But the invention also proposes a filtering material, a device for decontamination by filtration and a method for the production of a filtering material of this type that is capable of being carried out on an industrial scale for mass production.

To this end, the invention concerns a filtering material that is permeable to air and impermeable to human oral, nasal and/or ocular aqueous liquid excretions—in particular impermeable to human liquid excretions that are vectors for infectious agents such as viral particles—comprising at least one wad, termed a hydrophobic wad, of at least two hydrophobic sheets that are superimposed and each formed by porous paper with a grammage of less than 30 g/m²—in particular less than 20 g/m²—preferably comprised between 10 g/m² and 30 g/m², more preferably comprised between 10 g/m² and 20 g/m², each hydrophobic sheet being formed by cellulose fibers, termed cross-linked cellulose fibers, bonded together by means of hydrogen bonds and by means of covalent bonds formed with at least one group of cross-linking atoms, characterized in that at least a portion—in particular the entirety—of the hydroxyls of said cross-linked cellulose fibers that are not involved in the hydrogen bonds and that are accessible to gases—in particular accessible to air—form a covalent bond with an acyl group containing a hydrophobic chain.

The inventor has noticed that a single sheet of porous paper, with a grammage of less than 30 g/m² and formed by said cross-linked cellulose fibers and in which at least a portion of the hydroxyls of said cross-linked cellulose fibers that are not involved in the hydrogen bonds and that are accessible to gases form a covalent bond with an acyl group containing a hydrophobic chain, is not sufficiently watertight to be able to constitute a filtering material that is impermeable to human oral, nasal and/or ocular liquid excretions, which are vectors for infectious agents. In fact, the inventor has shown that a single hydrophobic sheet of this type cannot be used to retain a quantity of water—in particular several milliliters or tens of milliliters of water—deposited on the upper face of this sheet and that the entirety of this quantity of water passes through this single hydrophobic sheet instantaneously or over a few seconds, in particular when it is brought into contact with a hydrophilic surface.

However, the inventor has discovered that, in a completely unforeseeable manner, superimposing at least two—in particular two—of these very hydro-phobic sheets of porous paper, with a grammage of less than 30 g/m² and formed by said cross-linked cellulose fibers and in which at least a portion of the hydroxyls of said cross-linked cellulose fibers that are not involved in the hydrogen bonds and that are accessible to gases form a covalent bond with an acyl group containing a hydrophobic chain, can effectively retain a quantity of water, without leaking, over several hours or even several days—in particular several milliliters or tens of milliliters of water—deposited on the upper face of this wad of sheets, even when the wad of hydrophobic sheets is brought into contact with a hydrophilic surface and any possible loss of water is in fact due to evaporation.

Superimposing at least two hydrophobic sheets of this type means that a filtering material can be formed that is permeable to air and that is completely impermeable to human oral, nasal and/or ocular aqueous liquid excretions which carry pathogenic infectious particles such as viral particles.

Such a superimposition of two hydrophobic sheets formed by porous paper with a low grammage means that a stack of superimposed porous sheets can be formed that in particular is impermeable to respiratory liquid aqueous excretions that could be emitted by chronic exhalation or by sharp exhalation, in particular during a sneeze or a cough. The filtering material in accordance with the invention constitutes a filtering material for protection against infectious agents, in particular pathogenic infectious agents.

The hydrophobic property of an acylated hydrophobic sheet of this type formed by porous paper with a low grammage is visualized by measuring the contact angle formed between the principal plane of the hydrophobic sheet and a droplet of pure water deposited on the surface of the hydrophobic sheet. Typically, the value for the contact angle of the water droplet on the hydrophobic sheet is comprised between 90° and 180°, in particular more than 120°, preferably more than 150°. Furthermore, a hydrophobic sheet of this type has the property of being capable of maintaining a contact angle of this type with a droplet of pure water for a period of at least 4 hours.

Advantageously, the filtering material in accordance with the invention has sufficient flexibility to be pleasant in contact with the face.

In accordance with some advantageous embodiments, the filtering material in accordance with the invention comprises at least one wad, termed a hydrophilic wad, of at least one sheet formed by porous hydrophilic paper, with a grammage of less than 30 g/m²—in particular less than 20 g/m²—preferably comprised between 10 g/m² and 30 g/m², more preferably comprised between 10 g/m² and 20 g/m², said at least one sheet of porous hydrophilic paper being formed by fibers of cellulose bonded together at least by means of hydrogen bonds—and if appropriate by means of covalent bonds formed with at least one group of cross-linking atoms;

said at least one hydrophobic wad and said at least one hydrophilic wad being superimposed one upon the other in a manner such as to form a stack of superimposed porous sheets that is suitable for a flow of air that is to be filtered to pass through, by which means the air of the flow of air passing through the filtering material is filtered;

and in which at least one hydrophilic wad forms a principal free face of the stack of superimposed porous sheets, said free principal face being a free face for receiving the flow of air that is to be filtered.

The filtering material in accordance with the invention is completely impermeable to liquids, and in particular to water, and is completely permeable to air in the atmosphere. Not only can the filtering material in accordance with the invention be used to prevent—in particular to completely prohibit—human liquid excretions that are vectors for infectious agents, such as viral particles, from passing through it, but also, highly advantageously, it can retain and trap said human liquid excretions that are vectors for infectious agents on the filtering material. Thus, the destruction of the filtering material after use also means that infectious agents trapped on the filtering material are destroyed. The filtering material in accordance with the invention can be used to retain these infectious agents during the period of use of a device for the decontamination of air comprising such a filtering material by preventing dissemination of the infectious agents. The hydrophilic sheets) can be used to prevent saturation of the porous hydrophobic sheets) by the aqueous excretions.

In accordance with certain embodiments of a filtering material in accordance with the invention, at least one group of cross-linking atoms bonded to at least one cellulose fiber by means of covalent ether bonds has the following formula [Chem 1]:

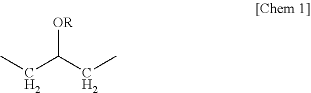

in which R is selected from a hydrogen atom (H) and an acyl group containing a hydrophobic chain.

In accordance with certain embodiments, at least one group of cross-linking atoms is formed by epichlorhydrin. It is entirely possible for at least one group of cross-linking atoms to be formed by an agent for cross-linking cellulose fibers that is distinct from epichlorhydrin, for example from an agent for cross-linking cellulose fibers that is used in the paper industry.

In accordance with the invention, the cellulose fibers of each hydrophobic sheet are cross-linked. The inventor has observed that not only does cross-linking of the cellulose fibers of a hydrophilic cellulose sheet not oppose subsequent acylation of the cellulose fibers cross-linked in this manner, but also that this cross-linking combined with acylation can in fact restrict the rotational mobility of the cross-linked and acylated cellulose fibers, increase the mechanical strength of the hydrophobic sheet—in particular the mechanical strength under wet conditions (“wet-strength”)—and maintain its “barrier” properties against aqueous respiratory liquid excretions over long periods.

In accordance with certain advantageous embodiments of a filtering material in accordance with the invention, at least one sheet of porous hydrophilic paper is formed by native and pure cellulose. However, in accordance with certain other advantageous embodiments of a filtering material in accordance with the invention, it is entirely possible for at least one sheet of porous hydrophilic paper to be formed by cross-linked cellulose fibers.

The hydrophobic sheets can provide the filtering material and the device for the decontamination of air by filtration with “barrier” properties and can maintain these “barrier” properties over time, including after exposing the filtering material to moisture. They can provide the filtering material with properties of permeability to gaseous fluids—in particular to atmospheric air—permitting breathing. They can also provide the filtering material with mechanical strength properties that are at least preserved—or even improved—compared with the mechanical properties of hydrophilic sheet(s) that have not been exposed to aqueous contamination.

Advantageously, the filtering material in accordance with the invention can be used to prevent individuals who are contagious but who wear a device for the decontamination of air by filtration comprising such a filtering material from contaminating healthy individuals who are riot wearing such a device; it can also be used to prevent healthy individuals who are wearing such a device from being contaminated. Clearly, advantageously, it is preferable for the contagious individuals and the healthy individuals to wear a device for the decontamination of air by filtration comprising a filtering material in accordance with the invention. The filtering material and the device for the decontamination of air by filtration are adapted to enable individuals to be protected against the transmission of viral particles transported in human oral, nasal and/or ocular aqueous liquid excretions.

In accordance with certain advantageous embodiments of a filtering material in accordance with the invention, the acyl group containing a hydrophobic chain is an acyl group containing a hydrophobic hydrocarbon chain. The expression “acyl group” designates any group of atoms with chemical formula R—CO—, in which R is a hydrophobic chain. R may be a hydrocarbon group containing 16 to 30 carbon atoms. Advantageously, at least one—in particular each—hydro-carbon group is a saturated hydrocarbon group. Advantageously, at least one—in particular each—acyl group containing a hydrophobic chain is selected from the group formed by a palmityl group (CH₃—(CH₂)₁₄—CO—), a stearyl group (CH₃—(CH₂)₁₆—CO—) and a behenyl group (CH₃—(CH₂)₂₀—CO—). It is entirely possible for at least one—in particular each—hydrocarbon group to be an unsaturated hydrocarbon group.

In certain embodiments of a filtering material in accordance with the invention, each of the two free faces of the stack of superimposed porous sheets is formed by a hydrophilic wad, at least one hydrophobic wad being interposed between the hydrophilic wads forming the two free faces of the stack of superimposed porous sheets. Advantageously, the porous hydrophobic sheet(s) and the porous hydrophilic sheet(s) form a pouch for stopping and retaining excretions in the form of aqueous microdroplets or macrodroplets in suspension in the inhaled/exhaled air. In these embodiments in accordance with the invention, the filtering material comprises an assembly of at least one hydrophobic wad, each of the two opposed principal faces of the assembly of hydrophobic wad(s) extending and facing an assembly of at least one hydrophilic wad. Each of the assemblies of hydrophilic wad(s) can be used to absorb aqueous excretions on both one and the other of the two principal faces of the filtering material, limiting or even completely annihilating any effect of rebound of the aqueous composition onto the assembly of porous hydrophobic sheet(s) and maintaining the barrier effect with maximum efficiency.

Advantageously, at least one—in particular each—hydrophobic sheet is formed by a portion of the thickness of a disposable paper tissue—in particular of the “Kleenex®” type (Kleenex®, Kimberly-Clark Corporation), the cellulose of said portion of the thickness of the disposable paper tissue being at least partially acylated. The inventor has determined that such a disposable paper tissue is in fact formed by a plurality of—in particular four—superimposed sheets of porous paper with a low grammage and that covalent grafting of acyl groups containing hydrophobic chains can be used to facilitate separation of these hydrophobic sheets, making it possible to produce a filtering material that is impermeable to liquid excretions and which has a permeability to air that remains compatible with optimal ventilation. Advantageously and in accordance with the invention, said hydrophobic wad comprises 2 to 4 hydrophobic sheets formed by 2 to 4 hydrophilic sheets of porous paper with a low grammage of a disposable paper tissue. The filtering material in accordance with the invention can therefore be made from a readily accessible starting material.

Advantageously and in accordance with the invention, at least one—in particular each—sheet of the stack of superimposed porous sheets is formed by a portion of the thickness—in particular a unitary constituent sheet—of a disposable paper tissue in particular of the “Kleenex®” type.

In accordance with certain embodiments of a filtering material in accordance with the invention, at least two sheets of said hydrophobic wad are assembled by means of at least one material, termed the assembly material, which is a permeable solid comprising at least one thermoplastic polymer. In accordance with certain embodiments of a filtering material in accordance with the invention, at least one hydrophobic wad is assembled with at least one hydrophilic wad by means of at least one material, termed the assembly material, which is a solid comprising at least one thermoplastic polymer. Advantageously, this assembly is made by means of fusion and solidification of said assembly material interposed between at least one porous hydrophobic sheet and at least one porous hydrophilic sheet. Advantageously, each thermoplastic polymer is permeable to air. Advantageously, each sheet of the stack of superimposed porous sheets is assembled with each of its adjacent sheets by means of at least one material, termed the assembly material, which is a permeable solid comprising at least one thermoplastic polymer.

In accordance with certain embodiments of a filtering material in accordance with the invention, at least one thermoplastic polymer is selected from the group formed by polyethylenes, polypropylenes, polyamides and poly-L-lactic acids. Advantageously, at least one thermoplastic polymer is selected so as to be capable of assembling at least one porous hydrophilic sheet and at least one porous hydrophobic sheet between which the assembly material is disposed, this assembly possibly being produced by the simple application of a hot surface—in particular the sole plate of an electric iron—in contact with one or the other of the porous hydrophilic or hydrophobic sheets.

It is entirely possible for said assembly material to be formed by a cold bonding assembly material. Advantageously, said cold bonding assembly material may be cellulose acetate.

In accordance with certain advantageous embodiments of a filtering material in accordance with the invention, the thermoplastic polymer extends only at the peripheral border of the stack of superimposed porous sheets. Advantageously, the central portion of the stack of porous superimposed sheets is free from assembly material, Thus, it is completely permeable to atmospheric air and allows substantially normal ventilation and respiration. The central portion of the stack of superimposed porous sheets is flexible and soft to the touch and biocompatible with the skin. It is entirely possible for the assembly material to reinforce the stiffness at the periphery of the stack of superimposed porous sheets. The assembly material may be used to participate in shaping the stack of superimposed porous sheets to adapt it to the morphology of the face of the individual wearing the filtering material.

Advantageously, the filtering material in accordance with the invention can be incinerated. It is adapted for destruction by incineration, without producing toxic substances other than CO2 and water, by which means the viral particles that can be retained in the filtering material in accordance with the invention are destroyed.

The invention also concerns a device for the decontamination of air by filtration, comprising
a filtration means comprising a filtering material in accordance with the invention, and;
means for adjusting this filtration means on an individual.

Advantageously and in accordance with the invention, the dimensions of the filtration means are such as to be able to cover at least the mouth and the nose of an individual wearing the device for the decontamination of air.

Advantageously, the filtration means may be in the form of a substantially conical funnel. Such a shape is particularly well suited to optimizing the effective filtration surface area of the filtration means. In practice, at least one porous hydrophilic sheet and/or at least one porous hydrophobic sheet may be formed by a “coffee filter” type cellulose device.

In certain embodiments of a device for the decontamination of air by filtration in accordance with the invention, the adjustment means comprise means for adjustment that can be undone, and/or a tie of elastic material.

In certain embodiments, the device in accordance with the invention is a device in the form of a mask, a hood, a balaclava, a ski mask, a shirt, a tunic, or a tissue.

In other embodiments, the device for decontamination by filtration is in the form of a protective glove.

In accordance with certain embodiments, the filtration means of the device for the decontamination of air by filtration is in the form of a flexible flat piece which is thin. It is entirely possible for the filtration means to have folds, in particular accordion folds, enabling the filtration surface area and comfort in use to be increased. The folding may be carried out by adding strips of absorbent paper in particular filter paper which can provide the filtration means with stiffness. It is entirely possible for such strips of absorbent paper to be used in order to provide the filtration means with any specific shape. It is entirely possible for a flexible non-elastic strip of material to be used in order to allow for adjustment and to keep the air decontamination device on the individual's nose.

The filtering material and the device for the decontamination of air in accordance with the invention have incineration capability properties that are substantially identical to the incineration capability properties of cellulose fibers.

The invention also concerns a method for the production of a filtering material in accordance with the invention, in which:
at least two first sheets are selected, each formed by porous paper with a grammage of less than 30 g/m², each first sheet being formed by cellulose fibers, termed cross-linked cellulose fibers, bonded together by means of hydrogen bonds and by means of covalent bonds formed with at least one group of cross-linking atoms;
said at least two first porous sheets undergo a reaction for the acylation of the cellulose fibers in a manner such as to form at least two first porous hydrophobic sheets, during which;
   each first sheet is impregnated with at least one fatty acid chloride—in particular at least one palmitic acid chloride (CH₃—(CH₂)₁₄—CO—Cl), at least one stearic acid chloride (CH₃—(CH₂)₁₆—CO—Cl) and at least one behenic acid chloride (CH₃—(CH₂)₂₀—CO—Cl), then;
   a flow of a gaseous composition—in particular a flow of atmospheric air—that is heated to a temperature that can enable said acylation reaction to take place between the cellulose of each first sheet and at least one fatty acid chloride in the gaseous state is applied to each first sheet, by which means at least two porous hydrophobic sheets formed by cellulose fibers are formed:
      which are bonded together by means of hydrogen bonds and by means of covalent bonds formed with at least one group of cross-linking atoms; and
      in which at least a portion—in particular the entirety—of the hydroxyls that are not involved in a hydrogen bond or in a covalent bond with said at least one group of cross-linking atoms form a covalent bond with an acyl group containing a hydrophobic chain; then
   said at least one hydrophobic wad of filtering material is formed by stacking said at least two first porous hydrophobic sheets.

In a method for the production of a filtering material in accordance with the invention, at least one porous hydrophobic sheet is formed by a reaction for the covalent grafting of at least one fatty acid chloride in the gaseous state onto the cellulose fibers of a porous hydrophilic sheet.

A reaction of this type may be obtained by bringing a solution of long chain fatty acid chloride in an apolar solvent, in particular a solvent selected from the group formed by petroleum ether, white spirit and ethyl ether, into contact with at least one porous hydrophilic sheet. This may be contact by immersing (a) porous hydrophilic sheet(s) in the solution of fatty acid chloride. This may be contact by nebulization of the fatty acid chloride solution onto the porous hydrophilic sheet(s). Next, a flow of gaseous composition, in particular a flow of air, is applied to the porous hydrophilic sheet(s) loaded with fatty acid chloride heated to a temperature that permits diffusion of at least a portion of the fatty acid chloride in the gaseous state over at least a portion of the thickness of the porous hydrophilic sheet(s) and a reaction of the fatty acid chloride in the gaseous state with the accessible free hydroxyl groups of the cellulose fibers of the porous hydrophilic sheet(s). A catalyst is not necessary. No catalyst is used. The hydrochloric acid formed as a result of the grafting reaction is transported by the flow of gaseous composition. This results in homogeneous grafting of fatty acyl groups onto the entirety of the free surface of the porous hydrophilic sheet(s) which become (a) porous hydrophobic sheet(s) because of the grafting. No qualitative or quantitative error in the grafting is possible. Advantageously, the reaction of each fatty acid chloride on the porous hydrophilic sheet(s) is biocompatible, i.e. the acylated cellulose fibers formed because of the grafting reaction are compatible with use of the filtering material in contact with and/or in the proximity of the airways of an individual wearing the decontamination device.

Grafting of the acyl groups containing a hydrophobic chain does not substantially affect the mechanical properties of the porous hydrophobic sheet(s) compared with the mechanical properties of the porous hydrophilic sheet(s). It does not substantially modify the flexibility and strength. It does not substantially modify the porosity. It also does not affect the permeability to air. The porous hydrophobic sheet(s) are permeable to a flow of gaseous composition directed substantially orthogonally to the plane of the porous hydrophobic sheet(s). In contrast, they are completely impermeable to aqueous particles that might be transported by the flow of gaseous composition. They also have a wet strength that is increased with respect to the porous hydrophilic sheets.

In certain advantageous embodiments of a production method in accordance with the invention, commercially available products and reagents are used in order to carry out the acylation reaction by means of covalent grafting. This acylation reaction using finished cellulose products such as disposable paper tissues, for example, is carried out while dry and under economically competitive conditions.

In certain embodiments of a method for the production of a filtering material in accordance with the invention:
at least one second hydrophilic sheet is selected, formed by porous paper with a grammage of less than 30 g/m², each second sheet being constituted by cellulose fibers bonded together by means of hydrogen bonds and by means of covalent bonds formed with at least one group of cross-linking atoms;
said at least one hydrophilic wad is formed by stacking each second hydrophilic sheet; and
said at least one hydrophobic wad and said at least one hydrophilic wad are superimposed in a manner such as to form a stack of superimposed porous sheets such that at least one hydrophilic wad forms a free face of the stack of superimposed porous sheets and of filtering material.

In certain embodiments of a method for the production of a filtering material in accordance with the invention, a step for assembly—in particular a step for assembly by bonding—of at least two porous sheets of the stack of superimposed porous sheets is carried out by heat sealing with at least one material, termed the assembly material, which is a permeable solid comprising at least one thermoplastic polymer. An assembly step of this type by bonding is carried out, for example, by interposing a strip of a thermoplastic polymer—in particular a heat fusible polymeric material—over at least a portion of the peripheral zone of the hydrophobic sheet and/or of the porous hydrophilic sheet and heating the thermoplastic material to a temperature that is higher than the fusion temperature of the thermoplastic material. Advantageously, this step is carried out with a heated sole plate of an electric iron. Extremely surprisingly, the thermoplastic material can be used to assemble at least one porous hydrophobic sheet and at least one porous hydrophilic sheet that have opposed surface hydrophobic/hydrophilic properties. Advantageously, the thermoplastic material is a tape of thermoplastic material similar to that in routine use in the field of clothing manufacture and in particular in making hems.

In certain advantageous embodiments of a production method in accordance with the invention, each first sheet respectively formed by porous paper with a grammage of less than 30 g/m² and each second sheet respectively formed by porous paper with a grammage of less than 30 g/m² is formed by a portion of the thickness of a disposable paper tissue.

In certain advantageous embodiments of a production method in accordance with the invention, at least two first sheets each formed by porous paper with a grammage of less than 30 g/m² and at least one first sheet formed by porous paper with a grammage of less than 30 g/m² are formed by all or a portion of the thickness of a disposable paper tissue, in particular of the “Kleenex®” type.

In certain embodiments of a method for the production of a filtering material in accordance with the invention carried out on an industrial scale, each first sheet is impregnated with a solution of at least one fatty acid chloride in an apolar solvent then, after evaporation of the apolar solvent, each first sheet is heated with a flow of gaseous composition in a manner such as to enable said reaction for the acylation of cellulose fibers by means of at least one fatty acid chloride containing a hydrophobic chain and in the gaseous state to take place on each first sheet and enable the formation of a porous hydrophobic sheet.

In certain embodiments of a method for the production of a filtering material in accordance with the invention carried out on an industrial scale, the acid chloride containing a hydrophobic chain is deposited (without an apolar solvent) on the surface of a porous hydrophilic sheet using any known printing technique.

In a method for the production of a filtering material in accordance with the invention carried out manually, a grafting solution is prepared comprising at least one fatty acid chloride diluted in a volatile aprotic solvent such as petroleum ether (commercially available in the form of “white spirit”), then the porous hydrophilic sheet to be treated is soaked with the grafting solution, then the volatile solvent is allowed to evaporate in the open air, but preferably under an extractor hood or in an open space. Next, a flow of hot air—for example generated by a hair dryer—is applied to the porous hydrophilic sheet in a manner such as to allow the fatty acid chloride in the gaseous state to react and to enable it to graft onto the porous hydrophilic sheet which is transformed into a porous hydrophobic sheet because of this grafting, and any excess reagent is eliminated.

The invention also concerns a filtering material, a device for the decontamination of air and a method for the production of a filtering material of this type characterized, in combination or otherwise, by some or all of the features mentioned above or below. Irrespectively of the formal presentation given, unless explicitly indicated otherwise, the various features mentioned above or below should not be considered to be strictly or inextricably linked together; the invention may concern only one of these structural or functional features, or only a portion of these structural or functional features, or only a portion of one of these structural or functional features, or in fact any grouping, combination or juxtaposition of some or all of these structural or functional features.

Other aims, features and advantages of the invention will become apparent from the following description, given by way of illustration, of certain embodiments made with reference to the accompanying drawings, in which:

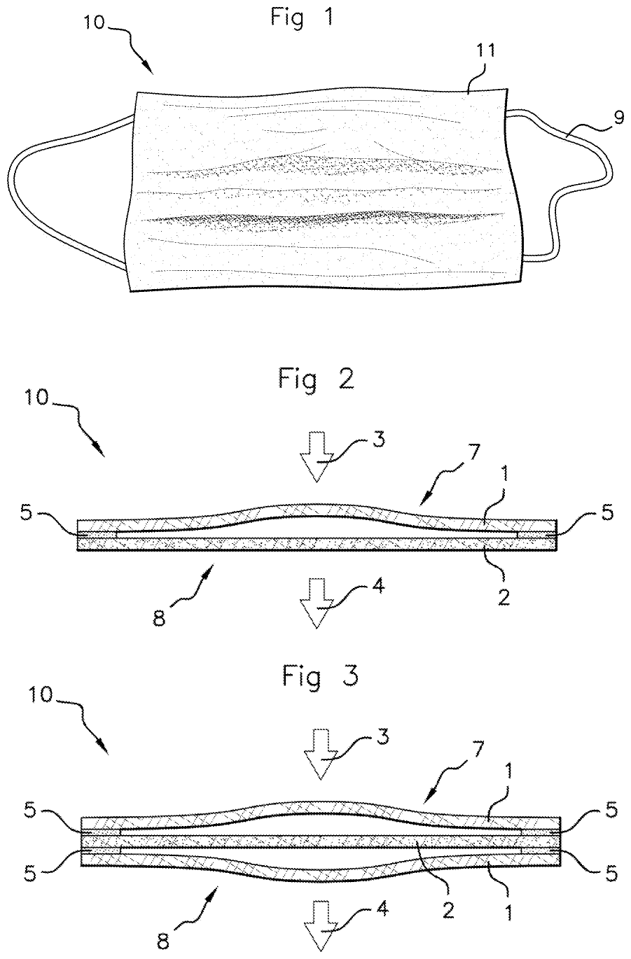

FIG. 1 is a representation of a first particular embodiment of a device for the decontamination of air in accordance with the invention;

FIG. 2 is a schematic representation in cross section of a first embodiment of a filtering material in accordance with the invention;

FIG. 3 is a schematic representation in cross section of a second embodiment of a filtering material in accordance with the invention, and;

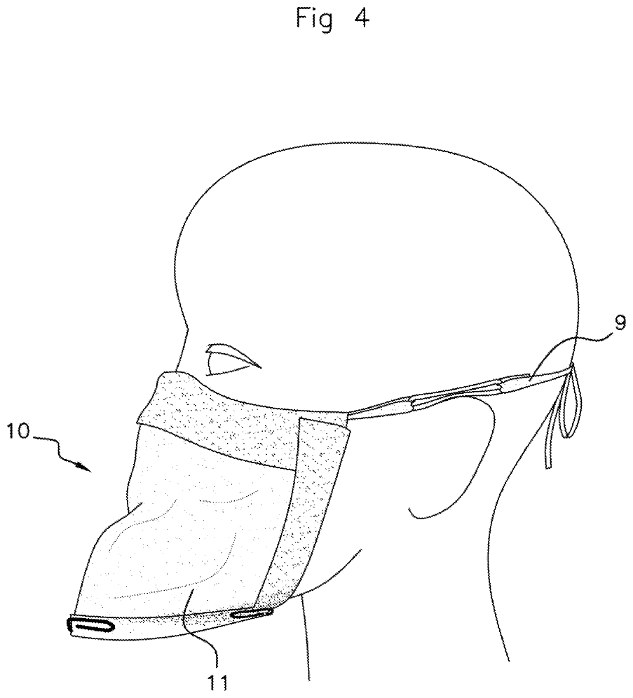

FIG. 4 is a representation of a second particular embodiment of a device for the decontamination of air in accordance with the invention.

Clearly, the dimensions and the proportions of the filtering materials and of the air decontamination device shown in FIGS. 1, 2 and 3 are not necessarily true to scale, for the purposes of clarity of the representation.

The device 10 for the decontamination of air shown in FIG. 1 is a respiratory protection mask dimensioned to be capable of covering the mouth and the nose of an individual. It comprises a filtration means 11 forming a filtering material in accordance with the invention, and means 9 for adjusting these filtration means 11 on an individual. The means 9 for adjusting the filtration means 11 are formed by two ties secured to the filtration means 11, enabling the filtration means 11 to be retained when applied to the face of an individual.

A schematic representation in cross section of a first embodiment of a filtering material of a device 10 in accordance with the invention for the decontamination of a flow of contaminated air 3 is shown in FIG. 2. The filtering material shown in FIG. 2 comprises a stack of a porous hydrophobic sheet 2 and a porous hydrophilic sheet 1. The porous hydrophobic sheet 2 and the porous hydrophilic sheet 1 are assembled by bonding by means of an assembly material 5 extending at the periphery of the stack of superimposed porous sheets. The assembly material 5 is formed by a thermoplastic polymer. Any other ways of assembling the porous hydrophobic sheet 2 and the porous hydrophilic sheet 1 are possible. It is possible to assemble the porous hydrophobic sheet 2 and the porous hydrophilic sheet 1 using staples. Not assembling the porous hydrophobic sheet 2 and the porous hydrophilic sheet 1 using specific means is also a possibility; the porous hydrophobic sheet 2 and the porous hydrophilic sheet 1 may be held in place by the individual wearing the device 10 for the decontamination of air.

The porous hydrophobic sheet 2 is formed by a disposable paper tissue modified by a reaction for covalent grafting of a fatty acid chloride. The porous hydrophobic sheet 2 is permeable to atmospheric air and completely impermeable to any aqueous composition, in particular impermeable to any aqueous composition loaded with infectious viral particles. The porous hydrophobic sheet 2 forms one of the two principal faces of the filtering material. The porous hydrophobic sheet 2 forms the downstream face 8 of the device 10 for the decontamination of air, i.e. the face of the device 10 for the decontamination of air via which a flow of decontaminated air 4 which can be breathed by the individual wearing the decontamination device 10 is emitted. It is entirely possible for the porous hydrophobic sheet 2 to be formed by only a portion of the stacked layers forming a disposable paper tissue and modified by a reaction for covalent grafting of a fatty acid chloride.

In this embodiment, the porous hydrophilic sheet 1 is an unmodified disposable paper tissue. The porous hydrophilic sheet 1 has absorbent properties so that the aqueous composition loaded with infectious viral particles retained by the porous hydrophobic sheet on the upstream principal face 7 of the decontamination device 10 is entirely retained in the porous hydrophilic sheet 1. It is entirely possible for the porous hydrophilic sheet 1 to be formed by only a portion of the stacked layers forming a disposable paper tissue.

In the first embodiment shown in FIG. 2, the porous hydrophobic sheet 2 forms one of the two principal faces of the filtering material. The porous hydrophobic sheet 2 forms the downstream face 8 of the device 10 for the decontamination of air, i.e. the face of the device 10 for the decontamination of air via which a flow of decontaminated air 4 which can be breathed by the individual wearing the decontamination device 10 is emitted. In this first embodiment shown in FIG. 2, the porous hydrophilic sheet 1 forms the other of the two principal faces of the filtering material. The porous hydrophilic sheet 1 forms the upstream face 7 of the device 10 for the decontamination of air, i.e. the face of the device 10 for the decontamination of air receiving the flow of contaminated air 3 by inhalation by the individual wearing the decontamination device 10.

The device 10 for the decontamination of air can be used to decontaminate a flow of contaminated air 3 transporting infectious viral particles and to form a flow of substantially decontaminated air 4 that is capable of being breathed by the individual wearing the decontamination device 10, without a risk of infection. However, the device 10 for the decontamination of air also and highly advantageously permits the infectious viral particles to be retained in the porous hydrophilic sheet 1 and/or in the space separating the porous hydrophilic sheet 1 and the porous hydrophobic sheet 2, avoiding contamination of persons moving around the individual wearing the device 10 for the decontamination of air, and because of this decontamination. Advantageously, the device 10 for the decontamination of air is intended to be destroyed and rendered inert after use. Destruction of this type is advantageously carried out by incineration.

A schematic representation in cross section of a second embodiment of a filtering material of a device 10 in accordance with the invention for the decontamination of a flow of contaminated air 3 is shown in FIG. 3. The filtering material shown in FIG. 3 comprises a stack of a first porous hydrophilic sheet 1 extending at the upstream face 7 of the decontamination device 10, a porous hydrophobic sheet 2 and a second porous hydrophilic sheet 1 extending at the downstream face 8 of the decontamination device 10, the porous hydrophobic sheet 2 being interposed between the first and second porous hydrophilic sheets. The upstream face 7 of the decontamination device 10 is intended to receive a flow of contaminated air 3 and the downstream face 8 is that one of the two principal faces of the device 10 for the decontamination of air from which a flow of decontaminated air 4 which can be breathed by the individual wearing the decontamination device 10 is emitted.

The porous hydrophobic sheet 2 and the porous hydrophilic sheets 1 are assembled by bonding by means of an assembly material 5 extending at the periphery of the stack of superimposed porous sheets and between the porous hydrophobic sheet 2 and each of the porous hydrophilic sheets 1. The assembly material 5 is formed by a thermoplastic polymer. Any other ways of assembling the porous hydrophobic sheet 2 and the porous hydrophilic sheet 1 are possible.

The porous hydrophobic sheet 2 is formed by a disposable paper tissue modified by a reaction for covalent grafting of a fatty acid chloride. The porous hydrophobic sheet 2 is permeable to atmospheric air and completely impermeable to any aqueous composition, in particular impermeable to any aqueous composition loaded with infectious viral particles.

In this embodiment, each porous hydrophilic sheet 1 is formed by an unmodified disposable paper tissue or by at least a portion of the thickness of a disposable paper tissue. Each porous hydrophilic sheet 1 has absorbent properties so that the aqueous composition loaded with infectious viral particles retained by the porous hydrophobic sheet 2 on one or the other of the two principal faces 7,8 of the decontamination device 10 is entirely absorbed by one or the other of the porous hydrophilic sheets 1 situated at the upstream face 7 or downstream face 8 of the decontamination device 10.

The device 10 for the decontamination of air can be used to decontaminate a flow of contaminated air 3 transporting infectious viral particles and to form a flow of substantially decontaminated air 4 that is capable of being breathed by a healthy individual wearing the decontamination device 10, without a risk of infection. The device 10 for the decontamination of air can also be used to decontaminate a flow of contaminated air that is transporting infectious viral particles emitted by an infected individual and who might contaminate their environment and to form a flow of substantially decontaminated air that can be inhaled by any healthy individual not wearing a decontamination device 10, without a risk of infecting that healthy individual.

However, the device 10 for the decontamination of air also and highly advantageously enables the infectious viral particles to be retained in one or the other of the first and second porous hydrophilic sheets 1 and/or in the space separating the porous hydrophobic sheet 2 and one or the other of the first and second porous hydrophilic sheets 1, preventing contamination of persons moving around the individual wearing the device 10 for the decontamination of air, and because of this decontamination.

The device 10 for the decontamination of air shown in FIG. 4 is a respiratory protection mask dimensioned to be able to cover the mouth, the nose and the chin of an individual. It comprises a filtration means 11 formed by a filtering material in accordance with the invention, and means 9 for adjusting these filtration means 11 on that individual. The means 9 for adjusting the filtration means 11 are formed by two ties attached securely to the filtration means 11, enabling the filtration means 11 to be retained when applied to the face of an individual.

Example 1—Impermeability to water of filtering material in accordance with the invention—Preparation of a porous hydrophobic sheet in accordance with the invention. The four secondary layers forming a disposable paper tissue (Kleenex®, Kimberly-Clark Corporation) were separated into two groups of two secondary layers with dimensions of 21 cm×21 cm square. Each group of two secondary layers formed a porous hydrophilic sheet in accordance with the invention. Each porous hydrophilic sheet formed by a disposable paper tissue had a respective mass of 1 g (22 g/m²). One of the porous hydrophilic sheets was immersed in a solution of stearic acid chloride in petroleum ether 100-150. The ratio of the mass of stearic acid chloride to the mass of petroleum ether 100-150 was 0.5%. The quantity of solution retained by the porous hydrophilic sheet was 1.5 g and the quantity of stearic acid chloride retained was 0.015 g, corresponding to 1.5% of the mass of the porous hydrophilic sheet. The major proportion of the solvent was allowed to evaporate spontaneously under an extractor hood and then the porous hydrophilic sheet was placed in a ventilated oven heated to 150° C. The reaction was allowed to continue for 2 minutes, by which means a porous hydrophobic sheet was formed by the disposable tissue. In the absence of a ventilated oven, it would have been entirely possible to use a. hair dryer to produce the hydrochloric acid formed during the reaction (by displacing the acylation reaction equilibrium), to heat the porous hydrophilic sheet, to form stearic acid chloride in the gaseous state and to enable the formation of the porous hydrophobic sheet by the reaction of stearic acid chloride in the gaseous state on the porous hydrophilic sheet.

By way of comparison, a porous hydrophilic sheet formed by two secondary layers of toilet paper with dimensions of 12 cm×10 cm and with a grammage of 41 g/m² was prepared. The mass of the porous hydrophilic sheet was 0.5 g. The porous hydrophilic sheet was immersed in a solution of stearic acid chloride in petroleum ether 100-150. The ratio of the mass of stearic acid chloride to the mass of petroleum ether 100-150 was 1%. The quantity of solution retained by the porous hydrophilic sheet was 0.8 g and the quantity of stearic acid chloride retained was 0.008 g, corresponding to 1.6% of the mass of the porous hydrophilic sheet. The major proportion of the solvent was allowed to evaporate spontaneously under an extractor hood and then the porous hydrophilic sheet was placed in a ventilated oven heated to 150° C. The reaction was allowed to continue for 2 minutes, by which means a porous hydrophobic sheet was formed by the toilet paper.

A first pouch, termed the tissue pouch, was prepared by suspending the porous hydrophobic sheet prepared from the tissue by its four corners. A second pouch, termed the toilet paper pouch, was prepared by suspending the porous hydrophobic sheet prepared from toilet paper by its four corners. The same quantity of water was poured into said tissue pouch and into said toilet paper pouch. Water did not leak instantaneously from either of the two pouches. Said tissue pouch appeared to be completely watertight over a period of more than 4 days. Said toilet paper pouch lost all of its water after 24 hours.

Example 2—Contact angle. A porous hydrophilic sheet formed by two of the four secondary layers forming a disposable paper tissue (Kleenex®, Kimberly-Clark Corporation) with dimensions of 21 cm×21 cm square was treated as described in Example 1. The porous hydrophilic sheet was treated with a solution of behenic acid chloride in petroleum ether 100-150. The ratio of the mass of behenic acid chloride to the mass of petroleum ether 100-150 was 0.75%. The temperature of the oven was raised to 160° C. A porous hydrophobic sheet was formed by a disposable paper tissue. A 200 μL droplet of distilled water was disposed on the surface of the porous hydrophobic sheet. The droplet remained in shape on the surface of the porous hydrophobic sheet without spreading. Thus, the contact angle was close to 180°.

Example 3—Mask for the decontamination of air by filtration. A porous hydrophobic sheet was prepared as described in Example 1. A porous hydrophilic sheet comprising a single secondary layer was also prepared from four secondary layers forming a disposable paper tissue (Kleenex®, Kimberly-Clark Corporation) with dimensions of 21 cm×21 cm square. The porous hydrophobic sheet and the porous hydrophilic sheet formed thereby were superimposed and a gauze of heat fusible polymer of the type used for making hems was interposed between the borders of the porous hydrophobic sheet and the porous hydrophilic sheet. The porous hydrophobic and hydrophilic sheets were assembled by heat sealing using a hot electric iron, The filtering material formed comprised a porous hydrophobic sheet formed by two secondary layers of a disposable paper tissue and one porous hydrophilic sheet formed by a single secondary layer of a disposable paper tissue. The filtering material in accordance with the invention was folded then stapled to the elastic elements at two of its opposed ends in order to form a mask for the decontamination of air by filtration in accordance with the invention. The decontamination mask was ready for use. It is possible to use it with the porous hydrophilic sheet directed towards the individual wearing the decontamination mask or with the porous hydrophobic sheet directed towards the individual wearing the decontamination mask, depending on whether protection of the individual from the environment is desired (porous hydrophilic sheet oriented towards the outside) or whether protection of the environment from the individual is desired (porous hydrophilic sheet oriented towards the individual).

Example 4—Variation of a mask for the decontamination of air by filtration in the form of a “duckbill”. A paper filtration device of the “coffee filter No. 4” type was selected. A large number of small holes was made in the paper in order to make the coffee filter porous while preserving its mechanical stiffness. The two outer faces of the coffee filter were covered with pieces of filtering material with appropriate dimensions as described in Example 1, by interposing a strip of heat fusible polymer gauze on the borders of the coffee filter between the coffee filter paper and the border of each piece of filtering material. Assembly was carried out by heat sealing with the aid of a hot electric iron, Next, elastic elements were stapled onto either side of the filter. The air decontamination mask was ready for use.

Example 5—Production of a filtering, watertight and absorbent tissue. A porous hydrophobic sheet was produced which comprised two secondary layers of a disposable tissue (Kleenex®, Kimberly-Clark Corporation) comprising four secondary layers, as described in Example 1. The porous hydrophobic sheet formed in this way was superimposed with a porous hydrophilic sheet formed by two secondary layers of a disposable paper tissue comprising four secondary layers. A strip of fusible gauze was interposed between the borders of the two porous hydrophobic and hydrophilic sheets and assembly was carried out by heat sealing, by application of a hot electric iron. The tissue was ready for use.

Example 6—Production of a second variation of a watertight and absorbent high filtration capacity tissue. The hydrophobic treatment as described in Example 1 was carried out on a complete tissue comprising four secondary layers in order to form a porous hydrophobic sheet comprising four secondary layers. The hydrophobic sheet formed was superimposed with a porous hydrophilic sheet formed by four secondary layers of paper from a disposable paper tissue. A strip of fusible gauze was interposed between the borders of the two porous hydrophobic and hydrophilic sheets and assembly was carried out by heat sealing by the application of a hot electric iron. A high filtration capacity tissue was ready for use.

Example 7—Production of a watertight and absorbent glove. Two pieces of filtering material as described in Examples 1, 3, 5 or 6 were prepared. Two hand shapes that were mirror images of each other were cut out with a die. A strip of heat fusible material gauze was interposed at the border of the cut pieces. Assembly was carried out by heat bonding by the application of a hot electric iron. As an alternative, it is entirely possible to use a 3D printer in order to deposit a strip of a heat fusible material completely around the shape.

Example 8—Manual production of a protective mask as illustrated in FIG. 4.

A piece of filtering material in accordance with the invention was prepared in the form of a disposable paper tissue forming an air filtration means 11. In a first step, one of the borders of the tissue was folded over in order to form a hem for receiving a section of a solid deformable material with low elasticity and a cord for adjustment of this filtration means on an individual. The low elasticity deformable solid section of material could be a copper wire, in particular a copper wire provided with a protective sheath. The diameter of the copper wire in cross section was preferably comprised between 1 mm and 1.5 mm in order to enable the protective mask to be retained on the nose of an individual. The adjustment cord may or may not be a textile elastic cord. The hem was sealed by heat sealing by means of a strip of heat fusible gauze. Any other means for sealing may be used, for example staples. Next, one of the two corners of the piece of filtering material opposite to the hemmed border was folded back onto the other by superimposing one of the two portions of the borders formed because of this folding onto the other. This superimposition was secured by longitudinal folding and locking the secured fold formed by means of staples or an assembly means of the paperclip type. It is entirely possible to secure this superimposition by bonding. The protective device was a mask in the form of a cone which fitted the face perfectly. The electrical cable allowed the mask to be adjusted and held on the nose. It was possible to adapt the depth of the mask by adjusting the longitudinal fold. A protective device was obtained that had a high efficiency in the filtration of human oral, nasal and/or ocular aqueous liquid excretions which could be obtained at low cost starting from a disposable paper tissue.

A number of variations and applications other than those described above may form the subject matter of a number of variations. In particular, it is clear that unless indicated otherwise, the various structural and functional features of each of the embodiments described above must riot be considered to be combined and/or closely and/or inextricably linked one to the other but, in contrast, should be considered to be simple juxtapositions. Furthermore, the structural and/or functional features of the various embodiments described above may form the subject matter as a whole or in part of any different juxtaposition or any different combination.

1. A filtering material that is permeable to air and impermeable to human oral, nasal and/or ocular aqueous liquid excretions, comprising: at least one wad, termed a hydrophobic wad, of at least two hydrophobic sheets that are superimposed and each formed by porous paper with a grammage of less than 30 g/m², each hydrophobic sheet being formed by cellulose fibers, termed cross-linked cellulose fibers, bonded together by means of hydrogen bonds and by means of covalent bonds formed with at least one group of cross-linking atoms,
wherein at least a portion of the hydroxyls of said cross-linked cellulose fibers that are not involved in the hydrogen bonds and that are accessible to gases form a covalent bond with an acyl group containing a hydrophobic chain.

2. The material as claimed in claim 1, wherein said material comprises at least one wad, termed a hydrophilic wad, of at least one sheet formed by porous hydrophilic paper with a grammage of less than 30 g/m², said at least one sheet of porous hydrophilic paper being formed by cellulose fibers bonded together at least by means of hydrogen bonds;
said at least one hydrophobic wad and said at least one hydrophilic wad being superimposed one upon the other in a manner such as to form a stack of superimposed porous sheets that is suitable for a flow of air that is to be filtered to pass through, by which means the air of the flow of air passing through the filtering material is filtered, and;
at least one hydrophilic wad forming a free principal face of the stack of superimposed porous sheets, said free principal face being a free face for receiving a flow of air that is to be filtered.

3. The material as claimed in claim 2, wherein each of the two free faces of the stack of superimposed porous sheets is formed by a hydrophilic wad, at least one hydrophobic wad being interposed between the hydrophilic wads forming the two free faces of the stack of superimposed porous sheets.

4. The material as claimed in one of claim 1, wherein at least two sheets of said hydrophobic wad are assembled by means of at least one material, termed the assembly material, which is a permeable solid comprising at least one thermoplastic polymer.

5. The material as claimed in claim 4, wherein at least one thermoplastic polymer is selected from the group formed by polyethylenes, polypropylenes, polyamides and poly-L-lactic acids.

6. The material as claimed in claim 4, wherein the thermoplastic polymer extends only at the peripheral border of the stack of superimposed porous sheets.

7. The material as claimed in one of claim 1, wherein at least one acyl group containing a hydrophobic chain is selected from the group formed by a palmityl group, a stearyl group and a behenyl group.

8. The material as claimed in claim 1, wherein each sheet of the stack of superimposed porous sheets is formed by a portion of the thickness of a disposable paper tissue.

9. A device for the decontamination of air by filtration, comprising:
a filtration means comprising a filtering material as claimed in claim 1, and;
means for adjusting these filtration means on an individual.

10. The device as claimed in claim 9, wherein the dimensions of the filtration means are such as to be capable of covering at least the mouth and the nose of an individual wearing the air decontamination device, the air decontamination device being in the form of a mask, a hood, a balaclava, a ski mask, a shirt, a tunic, or a tissue.

11. A method for the production of a filtering material as claimed in claim 1, in which:
at least two first sheets are selected, each formed by porous paper with a grammage of less than 30 g/m², each first sheet being formed by cellulose fibers, termed cross-linked cellulose fibers, bonded together by means of hydrogen bonds and by means of covalent bonds formed with at least one group of cross-linking atoms;
said at least two first porous sheets undergo a reaction for the acylation of the cellulose fibers in a manner such as to form at least two first porous hydrophobic sheets, during which;
each first sheet is impregnated with at least one fatty acid chloride;
a flow of a gaseous composition that is heated to a temperature that can enable said acylation reaction to take place between the cellulose of each first sheet and at least one fatty acid chloride in the gaseous state is applied to each first sheet, by which means at least two porous hydrophobic sheets formed by cellulose fibers are formed;
which are bonded together by means of hydrogen bonds and by means of covalent bonds formed with at least one group of cross-linking atoms; and
in which at least a portion—in particular the entirety—of the hydroxyls that are not involved in a hydrogen bond or in a covalent bond with said at least one group of cross-linking atoms form a covalent bond with an acyl group containing a hydrophobic chain; then
said at least one hydrophobic wad of filtering material is formed by stacking said at least two first porous hydrophobic sheets.

12. The method as claimed in claim 11, wherein:
at least one second hydrophilic sheet is selected, formed by porous paper with a grammage of less than 30 g/m², each second sheet being constituted by cellulose fibers bonded together by means of hydrogen bonds and by means of covalent bonds formed with at least one group of cross-linking atoms;
said at least one hydrophilic wad is formed by stacking each second hydrophilic sheet; and
said at least one hydrophobic wad and said at least one hydrophilic wad are superimposed in a manner such as to form a stack of superimposed porous sheets such that at least one hydrophilic wad forms a free face of the stack of superimposed porous sheets and of filtering material.

13. The method as claimed in claim 11, wherein a step is carried out for assembling at least two porous sheets of the stack of superimposed porous sheets by heat sealing with at least one material, termed the assembly material, which is a permeable solid comprising at least one thermoplastic polymer.

14. The method as claimed in claim 11, wherein each first sheet respectively formed by porous paper with a grammage of less than 30 g/m² and each second sheet respectively formed by porous paper with a grammage of less than 30 g/m² is formed by a portion of the thickness of a disposable paper tissue.

15. The method as claimed in claim 11, wherein the filtering material is shaped, by which means a device for the decontamination of air by filtration is formed.
